(19) United States
(12) Patent Application Publication
(10) Pub. No.: US 2007/0048233 A1
(43) Pub. Date: Mar. 1, 2007

(54) Artificial tanning composition containing medication

(75) Inventor: Alexander A. Bommarito, Freeland, MI (US)

Correspondence Address: JOHN MCCULLOCH, 5291 COLONY DRIVE NORTH, 1ST FLOOR, SAGINAW, MI, 48603, US

(73) Assignee: SPRAY TANNING, INC.

(21) Appl. No.: 11/509,350

(22) Filed: Aug. 24, 2006

Related U.S. Application Data
(60) Provisional application No. 60/711,474, filed on Aug. 26, 2005.

(51) Int. Cl. A61K 8/97; A61K 31/59; A61K 36/906

(52) U.S. Cl. 424/59; 424/74; 424/756; 514/167

(57) ABSTRACT

A topical, artificial skin tanning composition comprises a mixture of a tanning substance which darkens a person's skin and a medication which is active on and/or absorbable by such person's skin. The tanning substance may be dihydroxyacetone, the medication may be turmeric, and the mixture is one which may be sprayed or otherwise applied directly to a person's skin. Any objectionable color of the medication is masked by the tanning substance.

### CROSS REFERENCE TO RELATED APPLICATION

This application claims the benefit of the filing date of U.S. Provisional Application No. 60/711,474 filed Aug. 26, 2005.

This invention relates to a topical, artificial skin tanning composition comprising a mixture of a tanning solution which darkens a person's skin and a medication which is absorbable by the person's skin.

### BACKGROUND OF THE INVENTION

The use of topical, artificial skin tanning preparations and the use of topical medications are well known. It also is known that some topical medications, such as turmeric and extracts thereof are objectionable to many persons because they discolor the skin in a manner they consider to be unattractive. For example, turmeric products leave a distinctive, fairly bright yellow color on skin to which they have been applied. In addition, turmeric is an excellent dye and, as a consequence, it is not uncommon for clothing to become stained when worn by a person to whose skin turmeric has been applied.

Making medications available for topical application is desirable for many reasons, one of which is that many persons just do not like to take medication orally, even though the oral taking of such medications may be desirable. However, the same persons have no objection to the topical application of medications, and particularly if some positive social or cosmetic experience is achieved.

Thus, transdermal medication delivery can be medically beneficial upon delivery to a person's skin, as well as a solution to a person's reluctance to the taking of medication orally in accordance with a prescribed regimen.

The medical benefits of turmeric have been known for hundreds of years. Among these benefits are those resulting from treatments for burns, including sun burns, anti-inflammatory, antiseptic, antitumor, anti-allergic, and antioxidant causes, as well as cosmetic benefits such as nourishing and softening of the skin and reducing examples of aging, such as superficial wrinkling and scaling. However, the tendency of turmeric to discolor the skin yellow and to stain clothing inhibits the use of turmeric.

One of the desirable cosmetic effects sought by many people is that associated with tanning of the skin. Tanning can be achieved by exposure to the sun, by exposure to artificial tanning rays, and by the application to the skin of a skin-tanning solution or other preparation. Tanning by exposure to the sun or by exposure to artificial tanning rays is objected to by some persons because of inconvenience, thereby leaving the application of a tanning substance to the skin as the remaining alternative. However, artificial tanning substances currently available do not provide the user with any significant benefits, other than those associated with cosmetics. A tanning substance according to the invention provides not only the cosmetic benefits, but also therapeutic benefits because of the combination of the tanning substance and medications.

### SUMMARY OF THE INVENTION

One of the most commonly used solutions for artificial skin tanning is dihydroxyacetone (DHA). This substance can be combined with colorants or bronzers and other ingredients, such as moisturizers, antimicrobials, thickeners, solvents, emulsifiers, fragrances, surfactants, stabilizers, and pH adjusters. All of these ingredients are well known in the art.

A significant characteristic of the invention is the provision of a topical, foam, liquid, lotion, cream and the like composition comprising a mixture of the skin tanning substance and a medication which is active on or absorbable via the human skin. An appropriate composition is the aforementioned DHA and turmeric or an extract therefrom, such as curcumin. Both the DHA and the turmeric react with the skin and, after the passage of a few minutes from the application of the composition to a person's skin, the chemical reaction will cause the person's skin to darken and simulate tanning. The tanning solution will not react with the turmeric but will produce a deep tan coloring with no appearance of yellowing.

An appropriate, absorbable medication which may be included in the mixture is vitamin D. Good levels with local absorption of vitamin D can be achieved without adverse interactions.

If the composition also includes a coloring agent, such as NF caramel, which instantly colors a person's skin, it is easier to obtain a uniform application of the composition to the skin because the coloring of the skin will indicate to the applier where the solution has been applied, thereby minimizing the creation of streaks and different shades of coloring.

The use of NF caramel is preferred over other caramels because it provides initial masking of the turmeric yellowing and has no objectionable interaction with the medications.

### THE PREFERRED EMBODIMENT

A topical, artificial skin tanning composition according to the invention comprises a mixture of a tanning substance which darkens a person's skin and a medication which is active and may be locally or systemically absorbable by such person's skin. The tanning substance preferably comprises dihydroxyacetone (DHA) which may contain an appropriate amount of water and other additives such as a coloring agent capable of masking medications, biologics, and natural medicines. The medication preferably comprises turmeric or extracts therefrom, such as curcumin or curcuminoids. The mixture can be of such viscosity as to be sprayable in a fine mist from a spray bottle or the like. The mixture also can be in the form of a solution, cream, or lotion for topical application and should contain no insoluble particles such as would interfere with the activity on or absorbance by the skin. The mixture also should contain appropriate solvents for the other ingredients in the composition. Aqueous solvents are adequate for topical agents that easily penetrate the skin. Hydroalcohol solvents will provide better absorption for other agents.

The composition mixture may contain the tanning substance, DHA, with colorants to reveal application areas concurrently with application and assist in masking the color of medications, biologic, and natural medicines. Color additives can include NF caramel, at approximately 0.01% to 5%; yellow 5 food coloring at approximately 0.01 to 1%; blue 1 food coloring at approximately 0.01 to 3%; or red 4 food coloring at approximately 0.01% to 3%, or other natural colors as needed. All percentages are by volume.

The composition also can contain vitamin D at an approximate concentration of 100 IU to 2000 IU/ml.

The composition may contain turmeric or extracts thereof at approximately 0.1% to 15%, by volume, utilizing solvent extraction by means of water, ethanol, isopropyl alcohol, acetone, and their mixtures in all proportions or hydrodistillation or supercritical extraction using carbon dioxide and cosolvents.

If the tanning composition includes both vitamin D and turmeric products, the vitamin D should be at a concentration of about 100 IU to 2000 IU/ml and the turmeric products at the percentages specified.

The tanning composition can include other medications, such as biologic or natural medications, for topical use at therapeutic concentrations. For example, ibuprofen in a concentration of 10%-20% can be dissolved in a hydroalcohol base solution having a pH of about 5.0.

Natural medications, such as green tea, pomegranate, coffee beans, amino acids have been included in the composition at volume percentages from 0.01 to 6.0 with good results. The use of glutamine as an amino acid and pomegranate are common to the product. No reactions or breakdowns of DHA have occurred in stability testings at the preferred concentrations.

This disclosure is representative of presently preferred forms of the invention, but is intended to be illustrative rather than definitive thereof. The invention is defined in the claims.

I claim:

1. A topical, artificial skin tanning composition comprising a mixture of a tanning substance which darkens a person's skin and a medication which is locally or systemically absorbable by said person's skin.

2. The composition according to claim 1 wherein said medication comprises turmeric.

3. The composition according to claim 2 wherein said medication is curcumin extracted from turmeric.

4. The composition according to claim 1 wherein said tanning substance constitutes between about 0.5% and 15% by volume of said composition.

5. The composition according to claim 1 wherein said tanning substance is dihydroxyacetone.

6. The composition according to claim 1 wherein said mixture is in a sprayable liquid form.

7. The composition according to claim 1 wherein said mixture includes at least one vitamin.

8. The composition according to claim 7 wherein said vitamin is vitamin D.

9. The composition according to claim 8 wherein the content of said vitamin D is between 100-2000 IU/ml.

10. The composition according to claim 1 wherein said mixture is in non-sprayable form.

11. The composition according to claim 1 wherein said mixture is in lotion form.

12. The composition according to claim 1 wherein said mixture is in cream form.
